(19) United States
(12) Patent Application Publication
(10) Pub. No.: US 2011/0318403 A1
(43) Pub. Date: Dec. 29, 2011

(54) Preparation and Use of Pouteria Lucuma Extract

(75) Inventors: Ilya Raskin, Manalapan, NJ (US); Barbara Schmidt, Somerset, NJ (US); Caren Villano, Hillsborough, NJ (US); Leonel Eduardo Rojo Castillo, Brunswick, NJ (US)

(73) Assignee: Rutgers, The State University of New Jersey, New Brunswick, NJ (US)

(21) Appl. No.: 13/129,077

(22) PCT Filed: Nov. 12, 2009
(86) PCT No.: PCT/US09/64263

§ 371 Date: Sep. 9, 2011

Related U.S. Application Data
(60) Provisional application No. 61/199,029, filed on Nov. 12, 2008.

(51) Int. Cl. A61K 9/70; A61K 8/97; A61Q 17/04; A61Q 19/08; A61P 17/02; A61P 31/00; A61P 17/10; A61Q 19/00; A61K 36/185; C12N 5/071

(52) U.S. Cl. 424/443; 424/776; 424/59; 435/375; 424/74

(57) ABSTRACT

A method of preparing Pouteria lucuma extract was developed which comprises extracting oil from the nut. The lucuma nut oil (LNO) was effective to induce cell migration, enhance tissue regeneration and enhance wound healing. The lucuma nut oil provides an agent that is useful for therapeutic or cosmetic maintenance of skin and scalp.

### STATEMENT OF RELATED CASES

This application claims the priority benefit of provisional U.S. Patent Application No. 61/199,029 filed Nov. 12, 2008, which is incorporated herein by reference in its entirety.

### FIELD

The disclosure relates to plant extracts for therapeutic use. More particularly, the extracts are obtained from Pouteria species, e.g., Pouteria lucuma.

### BACKGROUND

Edible oils from natural sources are among the most consumed products in the food industry. Soybean, corn, and canola oils are among the most consumed worldwide, but little is known about the compositions of lesser known crops, including the presence and composition of any plant oils. This relative lack of knowledge is surprising, given that plant oils are important sources of polyunsaturated fatty acids (PUFAs) with health-promoting properties.

Lucuma, also called lucmo or eggfruit, is a tree fruit native to the highlands of western Chile, Peru, and southern Ecuador. Except for a few plantations in Costa Rica, lucuma is not cultivated outside of its endemic region. The fruits are ovate and yellow at maturity, with a dry yellow flesh. They can be eaten fresh, but commonly the fruit pulp is dehydrated and used as a flavoring in desserts and beverages. There are few published reports on bioactive components found in lucuma, except for those presenting the nutritional components of the fruit pulp.

It is commonly known that wound healing occurs as a multistage process. One paradigm of wound healing divides the process into an inflammatory phase, a proliferative phase, and a remodeling phase. Enhancing the body's natural wound healing process would be desirable to speed recovery times after injury. Moreover, faster wound healing would limit the time during which opportunistic infections can take hold, thereby reducing complications associated with skin wounds.

In addition to health concerns, humans also have cosmetic concerns related to their appearance and the image being projected. The skin, being the organ most visible to others, is a frequent source of cosmetic concerns. Consistent with the reality of cosmetic concerns, the cosmetics and skin care industries have long been significant segments of commerce and continue to grow as more effective cosmeceuticals are developed. Concomitant with that growth is the continuing escalation in research related to the cosmetics and skin care industries. Beyond skin scarring associated with improper or suboptimal wound healing, including burn healing, skin scarring is associated with such pervasive conditions as acne. Moreover, the natural aging process involves changes to the structure and function of skin that many view as imposing deleterious changes on one's image. Skin aging, for example, is associated with the undesirable loss of elasticity, hydration and color, among several other skin properties that, in the public's eye, change for the worse over time. A classic, undesired skin condition frequently viewed as a signal of advancing years is wrinkled skin. The efforts of the cosmetics/skin care industry to address these concerns has led to the increase in research noted above, as the industry struggles to provide the public with products that will reliably and effectively affect cosmetic appearance for the better.

Thus, there exists in the art a need to develop new therapeutic and/or cosmetic products and methods to increase the speed and efficacy of wound healing and/or to address cosmetic concerns with such skin conditions as scarring, aging, wrinkles, and loss of elasticity, among others.

### SUMMARY

The disclosure satisfies at least one of the aforementioned needs in the art in providing compositions comprising a plant extract therapeutically useful in therapeutic methods for healing purposes, such as wound healing and burn healing, and useful cosmetically to improve skin appearance (e.g., signs of skin aging, including wrinkled skin) and to maintain healthy skin as well as to repair skin blemishes, including acne. These compositions and methods contribute to significant savings in health and/or cosmetics costs while improving the healing processes and elevating the quality of life for substantial segments of the human population.

One aspect of the disclosure provides a method for preparing an extract from the nut of Pouteria lucuma, e.g., Pouteria lucuma O. Ktze, that comprises grinding the nut into a powder, extracting the nut powder with fluid and removing the fluid to obtain lucuma nut oil (LNO). In some embodiments, the method further comprises the step of preparing a pharmaceutical or cosmetic formulation containing the nut oil. In an embodiment of the method, the nut is first dried and then ground to the powder. In one embodiment the fluid used is selected from the group consisting of heptane and hexane (e.g., heptane) and, in a related embodiment, the fluid is removed by evaporation. In another embodiment, the fluid, e.g., heptane or hexane, is added to the nut powder at a ratio that is between 1 g:10 mL and 1 g:100 mL (nut weight:fluid volume). In some embodiments, the fluid, e.g., heptane or hexane, is added to the nut powder at a ratio of about 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, or 1:100 (weight:volume).

According to another aspect, an extract of Pouteria lucuma nut comprising the nut oil is provided. The disclosure comprehends a variety of fluids for preparing the lucuma nut extracts, which may result in dispersion, e.g., emulsion, or solvation of the lucuma nut oil. Preferably, the nut oil is at least partially dissolved in the fluid, e.g., solvent, used for extraction. Exemplary fluids include but are not limited to nonpolar organic compounds such as chloroform, acetone, acetonitrile, dichloromethane, ethyl acetate, alkanes such as heptane or hexane, and mildly polar organic compounds such as straight or branched, primary, secondary, or tertiary alcohols, e.g., isopropanol, methanol, ethanol, and glycol, as well as supercritical carbon dioxide. In some embodiments, the fluid is heptane.

According to another aspect, a phytomedicinal composition is provided that comprises a Pouteria lucuma nut extract comprising the nut oil. Phytomedicinal compositions in accordance with the disclosure comprise a lucuma nut extract as disclosed herein. Any fluid disclosed herein may be used in the extraction process in preparing a phytomedicinal composition, and any biocompatible fluid may be used in the phytomedicinal compositions, which are suitable for administration to living mammals, e.g., humans.

In another aspect, the disclosure provides a cosmetic formulation comprising the nut oil. In some embodiments, the cosmetic formulation comprises an antibiotic. In yet other embodiments, the cosmetic formulation comprises a skin health-promoting agent. The skin health-promoting agent may be Shea butter, an antioxidant, a retinoid, a moisturizer, a collagen-promoting agent, a sun screen, an alpha hydroxyl acid, jojoba oil, a silk extract, isopropyl palmitate, methyl paraben, and propyl paraben. The antioxidant may be vitamin A, vitamin C or vitamin E. The phytomedicinal composition and the cosmetic formulation can each be formulated as a cream, lotion, gel, paste, or spray for application.

Yet another aspect is a method for promoting wound healing comprising administering an effective dose of the phytomedicinal composition of the disclosure (LNO). In a related aspect, disclosed herein is a method of inducing cell proliferation by contacting a cell with LNO. In one preferred embodiment the cell is a fibroblast.

Another aspect is drawn to a method of enhancing tissue regeneration by contacting a tissue capable of regeneration with P. lucuma nut oil so that the tissue regenerates at a rate that is faster than is exhibited by the tissue in the absence of the nut oil. In a related aspect, the disclosure includes a method of promoting skin healing comprising contacting the skin with a formulation comprising the nut oil extract.

Still another aspect is a method of healing a skin burn comprising contacting the skin burn with a pharmaceutical composition comprising the extract of P. lucuma nut in an amount effective to promote skin healing in a patient in need thereof. In one aspect, the pharmaceutical composition further comprises a skin health-promoting agent. In another aspect, the pharmaceutical composition further comprises an antibiotic.

Yet another aspect is a method of reducing, eliminating or preventing a skin condition selected from the group consisting of acne, skin aging and wrinkled skin comprising contacting skin with a cosmetic formulation comprising the extract described herein, i.e., an extract comprising lucuma nut oil. In embodiments drawn to methods of reducing, eliminating or preventing skin aging or wrinkled skin, the cosmetic formulations according to the disclosure antagonize reduction in the expression of collagen and/or elastin, and may induce expression of collagen and/or elastin. In some embodiments, the cosmetic formulation further comprises an anti-acne composition and/or an antibiotic. In a related embodiment, the anti-acne composition may be salicylic acid, benzoyl peroxide, glycolic acid, sulfur, retinoic acid, peat water, resorcinol, silt, peat, permethin, azelaic acid, clindamycin, adapalene, erythromycin and sodium sulfacetamide.

In another aspect, the disclosure provides a dressing for the delivery of a composition of the disclosure, wherein the dressing comprises a composition comprising a lucuma nut oil. After application of the topical composition to the compromised tissue, the tissue may be covered with a dressing.

In yet another aspect, a transdermal patch is provided comprising a pad material having an upper surface and a lower surface, an adhesive on the lower surface, and a pharmaceutical composition comprising a lucuma nut oil on the lower surface. In a related embodiment, the patch further comprises an antibiotic.

Other features and advantages of the present disclosure will become apparent from the following detailed description. It should be understood, however, that the detailed description and the specific examples, while indicating preferred embodiments of the disclosure, are given by way of illustration only, because various changes and modifications within the spirit and scope of the disclosure will become apparent to those skilled in the art from this detailed description.

### BRIEF DESCRIPTION OF THE DRAWING

The following drawing forms part of the present specification and are included to further illustrate aspects of the disclosure. The disclosure may be better understood by reference to the figures of the drawing in combination with the detailed description of the specific embodiments presented herein.

FIG. 1 is a bar graph of migration of fibroblast cells in vitro.

FIG. 2 shows results from in vivo wound healing and regeneration models. FIG. 2A shows results from a wound healing model. FIG. 2B shows results when tailfin primordia of 5-day-old zebrafish larvae (n=6) were amputated just posterior to the notochord.

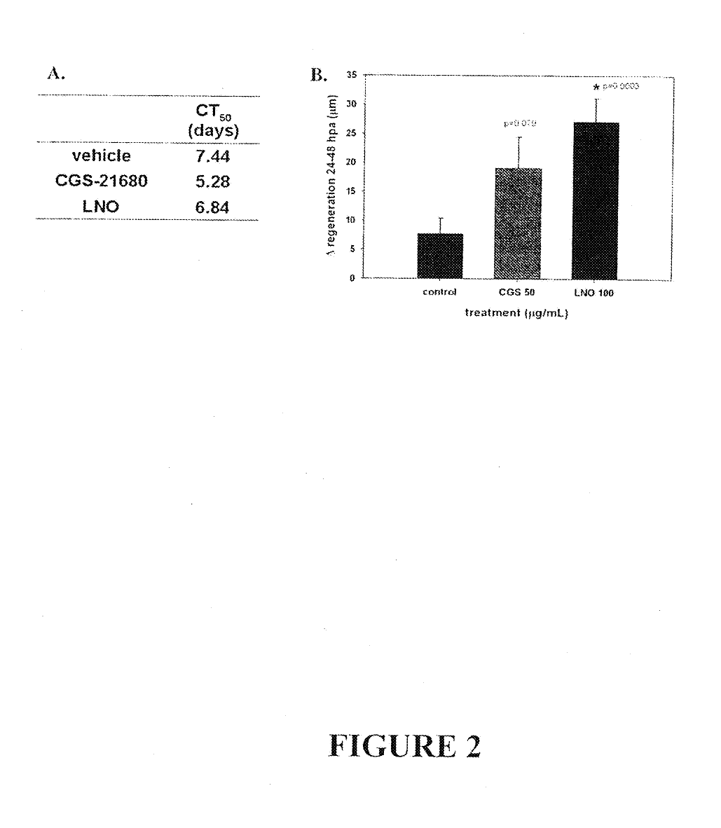

FIG. 3 provides a chromatographic profile of FAMEs from LNO analyzed by GC-MS. (A) Chromatograms of LNO 10 dilution, and (B) food industry standard mixture of 37 FAMEs, both LNO and standards were injected at 100 μg/mL.

FIG. 4 shows a time course of in vivo wound healing effect of LNO. The percent of wound closure was evaluated during 11 days of topical application of LNO 200, 500 and 1000 μg/mouse. Values are presented as mean±standard error of five replicates (*p<0.05 vs vehicle). One-way ANOVA followed by Dunnett's test was applied for comparisons between the treated and vehicle.

FIG. 5 shows the progression of the dermal wound healing induced by LNO. Representative images of the wound-healing progression in mice after daily application of LNO (500 and 1000 μg/mL), vehicle (carboxymethyl cellulose 50.5%, PBS pH7.4) and CGS (10 μg/mouse). Photos of the wounds were taken every other day during eleven days post-skin wounding (DPSW).

FIG. 6 illustrates the effect of LNO on tail fin regeneration and angiogenesis in transgenic zebrafish larvae. (A) Representative fluorescent micrographs of GFP-positive endothelial cells during tail fin regeneration induced by 100 μg/mL of LNO (LNO 100). B. Quantitation of tail endothelial cells regeneration 48 hours after amputation (hpa) of tail fin. Results were expressed as mean±standard error of nine replicates (*p=0.003, p=0.079 vs control).

FIG. 7 establishes the dose-dependent effect of LNO on tail fin endothelial repopulation of transgenic zebrafish larvae. The increase in fluorescent GFP-positive-endothelial cells 48 hours after amputation (hpa) of tail fin was dose-dependent. Results were expressed as mean±standard error of nine replicates.

FIG. 8 shows the effect of LNO on human fibroblasts migration. Human neonatal fibroblasts were marked with the green fluorescent probe Calcein-AM. The effect of LNO on fibroblast migration was evaluated using the ORIS™ cell migration assay as described in the Examples. Digital images were taken 72 hours after starting the experiment. From left to right: time zero (t=0), Vehicle, LNO 40 μg/mL (LNO 40) and LNO 60 μg/mL (LNO 60).

FIG. 9 illustrates the effect of LNO on cell migration in the scratched fibroblast assay. Scratched fibroblasts were treated with LNO 60 μg/mL (n=3) or with equivalent concentrations of pure fatty acids (FAs), oleic acid (OA, n=4), linoleic acid (LA, n=3) or 10% fetal bovine serum (FBS) as positive control. The effect of LNO and FAs were evaluated as described in the Examples. Values are the mean of four replicates±standard error (**p<0.01 vs FBS, One way ANOVA, Newman-Keuls Multiple Comparison Test).

FIG. 10 shows the effect of LNO on vinculin expression and stress fibers formation in fibroblasts. Human dermal fibroblasts were treated with LNO 60 μg/mL or vehicle and doubly stained with Rhodamine Phalloidin (red) and mAb anti-vinculin (green). LNO caused a mild decrease in stress fibers (F-actin) and increased the immunoreactivity to vinculin.

FIG. 11 establishes the effect of LNO on LPS-induced nitric oxide production in RAW 264.7 cells. RAW cells were pretreated with either vehicle alone or LNO for 2 hours. Subsequently, LPS (1 μg/mL) was added to each well and incubated for 8 hours. LPS significantly increased NO production in the non-stimulated cells (Control). LNO decreased LPS-induced NO production in a concentration-dependent manner. Results are expressed as mean±standard error (***p<0.001 vs LPS**p<0.01 vs LPS, One way ANOVA, Newman-Keuls Multiple Comparison Test).

FIG. 12 shows the effect of LNO on cellular senescence. Nuclei of non-senescent (control) and H₂O₂-induced senescent (H₂O₂ and H₂O₂+LNO) cells were stained with Acridine orange and photographed at 25× magnification. In control cells, most of the nuclei showed a non-senescent phenotype with classical elongated nuclear shape and homogeneous size.

FIG. 13 shows the effect of LNO on cellular senescence. These cells were observed under 100× magnification. Nuclear morphology of non-senescent dermal fibroblast was elongated (white arrows). Abnormal morphology (globular, folded, fragmented or oversized) was observed in senescent fibroblasts (orange arrows).

### DETAILED DESCRIPTION

The disclosure provides a method of preparing an extract from the nut, or seed, of the lucuma fruit. The active extract is useful for wound healing, for example, for treating trophic ulcers, surgical incisions, burns, abscesses, and the like. The use of a lucuma nut extract to treat such conditions, or to ameliorate or alleviate at least one symptom of such a condition, or to prevent or reduce the severity of such a condition, provides widely useful therapeutic forms that are readily prepared, relatively inexpensive and safe. The benefits to the individual are apparent in reducing the time taken for recovery from a wound, the expense of preventing or treating a wound, or ameliorating a symptom of a wound, and the time lost to wound recovery. To facilitate an understanding of the disclosure, the following express definitions are provided.

Disclosed herein is the biochemical composition of nonpolar organic (e.g., heptane) extracts of lucuma nut oil (LNO) and activity measures of LNO in several bioassays relevant to wound healing, including burn healing. GC-MS analyses of fatty acids methyl esters (FAMES) derived from LNO showed five major fatty acids, i.e., linoleic acid (38.9%), oleic acid (27.9%), palmitic acid (18.6%), stearic acid (8.9%) and γ linolenic acid (2.9%), which collectively account for 97.2% of the total oil content of the lucuma nut. An additional 16 fatty acids were found in minor concentrations. In a murine model of wound healing, LNO significantly enhanced cutaneous wound closure. LNO also promoted tail fin regeneration in transgenic zebrafish larvae 48 hours after amputation. Further, LNO significantly promoted migration of human fibroblasts and decreased LPS-induced NO production in macrophages. LNO-treated fibroblasts showed changes in the expression of vinculin and in the formation of stress fibers. LNO did not display anti-bacterial or anti-fungal effect. The results indicate that the wound healing and anti-inflammatory properties of LNO, with its unique fatty acid composition, are beneficially used in a variety of pharmacological and cosmeceutical applications.

The fruit of Pouteria lucuma O. Ktze, commonly named as “lucuma” in Chile and Peru, is widely consumed in these countries. Lucuma fruit consists of an edible fleshy outer part and a hard inner seed, or nut, that is inedible and that is usually discarded when the fruit is consumed or processed. Lucuma nut, however, is rich in oil, the composition of which has never been investigated. There is also virtually no information on the effects of lucuma on human health. This disclosure provides a description of the biochemical composition and pharmacological effects of Lucuma Nut Oil (LNO). Specifically disclosed herein are experimental results of comprehensive GC-MS analyses of fatty acids methyl esters from LNO and experimental results establishing the effect of LNO on inflammation, cell migration, angiogenesis, cellular senescence, bacterial and fungal growth and wound healing by using several in vivo and in vitro models.

As used herein, the term “effective dose” means a dose comprising an extract of P. lucuma that is able to cause a measurable change in wound healing time. The exact value of an effective dose varies based upon the sensitivity and size of each patient, and is readily determinable by one of skill in the art using conventional procedures for the routine administration of therapeutics. An effective dose of a Pouteria lucuma nut extract is between about 200 μg per wound (or topical application) per day to about 1,000 μg per wound (or topical application) per day. An exemplary effective dose is about 500 μg per wound/application per day.

The term “extract” as used herein means a substance or composition obtained from a plant or plant part source, regardless of whether the substance or composition is found external to the plant (i.e., an exudate), is found within the plant or plant part but external to the cells thereof, or is found within the cells of the plant. Chemical and/or physical action, as would be understood in the art, may be required to obtain the substance or composition from the plant or plant part.

The terms “pharmaceutically acceptable carrier” or “physiologically acceptable carrier” as used herein refer to one or more formulation materials suitable for accomplishing or facilitating the delivery of an extract of P. lucuma as a pharmaceutical composition.

The term “skin health-promoting agent” means any composition known in the art to decrease inflammation, reduce wrinkles, repair elasticity, repair or prevent discoloration, reduce or eliminate acne, improve skin tone, reduce or retard skin aging, reduce skin pore size, improve collagen production, replenish essential nutrients in the skin, improve skin hydration, reduce skin sagging, improve skin texture, or generally promote healthy skin. Such agents include, but are not limited to, Shea butter, antioxidants, retinoids, vitamins A, C, and E, Vaseline, aloe vera, lanolin, collagen-promoting agents, sun screens, alpha hydroxy acids, jojoba oil, silk extracts, isopropyl palmitate, methyl paraben and propyl paraben.

“Skin aging” has an accepted meaning in the art and is characterized by some or all of the following properties: Epidermis shows a progressive reduction in melanocytes and Langerhans cells decrease in density; Dermis becomes relatively acellular, avascular and less dense; nerves, microcirculation and sweat glands gradually decline leading to decreased thermoregulation and decreased sensitivity to burning, there is a progressive loss of elasticity, and an ever-increasing susceptibility to traumatic purpura or bruising; Subcutaneous layer of skin shows fat atrophy on the cheeks and extremities; decreased surface lipids, decreased hydration. The aging skin also shows ever-increasing discoloration and may show liver spots.

“Wrinkled skin” is given the meaning it has acquired in the art and refers to skin in which elasticity has been reduced to an extent that skin folds, or wrinkles, become increasingly apparent.

As used herein “patch” comprises a topical composition according to the disclosure and a covering layer, such that the patch can be placed over a wound, incision, or burn, thereby positioning the patch/composition adjacent to the compromised tissue surface. Preferably, the patch is designed to maximize composition delivery through the stratum corneum, upper epidermis, and into the dermis, and to minimize absorption into the circulatory system, reduce lag time, promote uniform absorption, and reduce mechanical rub-off.

Exemplary patches include (1) a matrix-type patch; (2) the reservoir-type patch; (3) the multi-laminate drug-in-adhesive-type patch; and (4) the monolithic drug-in-adhesive-type patch; (Ghosh, T. K., et al., Transdermal and Topical Drug Delivery Systems, Interpharm Press, Inc. p. 249-297 (1997), incorporated herein by reference). These patches are well known in the art and generally available commercially.

The term “dressing”, as used herein, means a covering designed to protect and or deliver a composition on or in the covering. “Dressing” includes coverings such as a bandage, which may be porous or non-porous, and various inert coverings, e.g., a plastic film wrap or other non-absorbent film. The term “dressing” also encompasses non-woven or woven coverings, particularly elastomeric coverings, which allow for heat and vapor transport. These dressings allow for cooling of the pain site, which provides for greater comfort.

The term “lucuma nut oil” or “LNO” is used interchangeably with “Pouteria oil” to refer to the therapeutic composition extracted from the nut of the fruit of a plant of the Sapotaceae family of plants, such as a plant of the Pouteria genus, e.g., Pouteria lucuma. The LNO comprises a plurality of compounds, at least one of which is therapeutically active in promoting wound or burn healing, in promoting regeneration or bioregeneration, and in delaying senescence.

### The Pouteria Plant

The therapeutic oil present in the compositions according to the disclosure is obtained from a plant in the Pouteria genus of plants or from a plant in a genus closely related to Pouteria, such as plants in the Manilkara genus. The Pouteria genus of plants is taxonomically organized within the Sapotaceae family of flowering plants, belonging to the order Ericales. Suitable plants serving as sources of the therapeutic oil include, but are not limited to, Pouteria species such as Eggfruit (Pouteria lucuma), Mamey (P. sapota), Abiu (P. caimito), Canistel (P. campechina), Green Sapote (P. viridis), Bully tree (P. multiforma), Curiola (P. torta), Cinnamon apple (P. hypoglauca), Frutao (P. ramiflora), Black apple (P. australis), P. sandwicensis, and species of the related Manilkara genus, e.g., Sapodilla (M. zapota van Royen), including its many cultivars, as well as Ausubo (M. bidentata), M. jaimiqui, and others.

### Pouteria Oil

The compositions according to the disclosure comprise a Pouteria oil, which is a therapeutically useful extract of the nut, or seed, of the fruit of the plant. The nut extract is referred to as an oil in a loose sense, and may include compounds that are not chemical oils, such as organic acids, carbohydrates, and the like. The nut extracts according to the disclosure do share the property of being at least partially soluble in nonpolar organic solvents, e.g., heptane or hexane and, for that reason, the extracts are loosely referred to as oils. Moreover, because many of the therapeutically beneficial nut extracts are obtained from species of the Pouteria genus, the term “Pouteria oil” is used in reference to such extracts, even though some plant extracts according to the disclosure are obtained from species of genera related to the Pouteria genus, e.g., the Manilkara genus. As noted elsewhere in this disclosure, “Pouteria oil” is also used interchangeably with “lucuma nut oil” or “LNO.”

The disclosure also provides for the therapeutic or cosmetic use of components of LNO and, in particular, any of the fatty acids components of LNO, such as linoleic acid, oleic acid, palmitic acid, stearic acid and γ linolenic acid, used in singular isolated form or in combinations of at least two such components, in the compositions according to the disclosure.

Several aspects of the disclosure provide for methods of inducing cell migration, promoting wound healing, including burn healing, promoting tissue regeneration and reducing, eliminating or preventing a skin condition such as acne, skin aging or wrinkled skin. In general, the methods comprise bringing a therapeutically or cosmetically effective amount of an LNO-containing extract, or purified components of the LNO (e.g., one or more fatty acids), into contact with a cell, tissue or organ, e.g., skin.

### Fluid

LNO is extracted from the nut or seed of a plant according to the disclosure using a fluid as described herein. Typically, the extraction fluid is a nonpolar organic compound or a mildly polar organic fluid that is liquid at room temperature. In some embodiments, the fluid is a solvent for LNO, but in other embodiments, the fluid is a medium into which the LNO is dispersible. Exemplary fluids for extraction of LNO are chloroform, acetone, acetonitrile, dichloromethane, ethyl acetate, ethanol, alkanes such as heptane or hexane, supercritical carbon dioxide, and straight or branched chain, primary, secondary or tertiary alcohols such as isopropanol, methanol, and ethanol.

The Pouteria Extraction Process

One method according to the disclosure is a method of preparing an extract of Pouteria lucuma, wherein lucuma nut is ground into a powder, the nut oil is extracted from the powder by a fluid, and the fluid is removed to obtain an oil. The raw material lucuma nut is ground, pulverized, broken, shredded, smashed, crushed, cracked and/or sieved to an appropriate size and shape. For example, a coffee grinder, hammer mill or similar size-reduction device, is used to reduce the lucuma nut to an optimum particle size for extraction of oil with a particular fluid. One suitable size range for nut particles upon grinding or pulverization is 750 μm to 1 mm diameter, although smaller and larger particles will be functional and are contemplated by the disclosure. A variety of fluids may be used to extract the lucuma nut oil, for example chloroform, acetone, acetonitrile, dichloromethane, ethyl acetate, ethanol, alkanes such as heptane or hexane, isopropanol, methanol, other alcohols, and supercritical carbon dioxide. The P. lucuma nut oil disclosed herein is extracted using a fluid such as heptane that provides extracted LNO that does not require further fractionation. In addition, a combination of compounds in any proportion may function as an extraction fluid, e.g., heptane and hexane, or heptane and chloroform. Following extraction, removal of the extracting fluid, where appropriate, is all that need be done to yield the therapeutic or cosmeceutical according to the disclosure.

The extraction process is typically conducted at room temperature, but the process is compatible with elevated temperatures in the range of 22° C. to 100° C., although it will frequently be conducted at 22° C. to 65° C. or from 22° C. to 50° C. or from 22° C. to 37° C. The extraction process is also typically conducted at a uniform pressure, e.g., atmospheric pressure, although elevated or reduced pressures are also contemplated for practice of the extraction methods. Thus, in certain embodiments of the extraction methods, pressurized fluid will be brought into contact with lucuma nut or nut meat, or the fluid and nut or nut meat will be in fluid communication within a pressurized vessel. In other embodiments, the nut or nut meat and fluid will be in fluid communication in a vessel under lowered pressure, e.g., partial or substantially complete vacuum.

Extracts according to the disclosure can be administered to mammals in an effective dose that is able to enhance wound healing. Alternatively, the extract can be administered to mammals in an effective dose that is able to enhance healing of skin burns. Other uses for the extracts of the disclosure include administration of a dose effective to eliminate, reduce, prevent, or reduce the likelihood of acne.

LNO-containing extracts may be suitable for use without further processing. Alternatively, raw extracts may be further processed to reduce the volume by any known technique, e.g., evaporation, including but not limited to rotary evaporation, lyophilization, filtration, and the like. Additional therapeutic compounds may also be included in the compositions according to the disclosure, with the composition comprising LNO and, e.g., excipients, carriers, binders, and the like, all of which are described below in addressing compositions and routes of administration of compositions comprising LNO. One exemplary embodiment illustrating the inclusion of an additional therapeutic compound relates to topical compositions that also include an anti-acne agent. Examples of anti-acne agents include, but are not limited to, salicylic acid, glycolic acid, benzoyl peroxide, sulphur, retinoic acid, peat water, resorcinol, silt, peat, permethin, azelaic acid, clindamycin, adapalene, erythromycin, sodium sulfacetamide, and combinations thereof. It is apparent that compositions according to the disclosure can also include more than two therapeutic compounds.

### Compositions and Routes of Administration

The pharmaceutical compositions and cosmetic formulations are administered by any route that delivers an effective dosage to the desired site of action, with acceptable (preferably minimal) side-effects. Suitable skin formulations may include topically acceptable liquids, creams, oils, lotions, ointments, gels, or solids, such as conventional cosmetic night creams, foundation creams, suntan lotions, sunscreens, hand lotions, make-up and make-up bases, masks and the like. The compositions can contain other optional suitable ingredients such as Vitamin A, C and E, alpha-hydroxy or alpha-keto acids such as pyruvic, lactic or glycolic acids, lanolin, Vaseline, aloe vera, methyl- or propyl-paraben, pigments and the like. Suitable topically acceptable carriers include water, petroleum jelly, petrolatum, mineral oil, vegetable oil, animal oil, organic and inorganic waxes, such as microcrystalline, paraffin and ozocerite wax, natural polymers, such as xanthanes, gelatin, cellulose, collagen, starch, or gum arabic, synthetic polymers, alcohols, polyols, and the like. In one embodiment, the carrier is a water-miscible carrier composition that is miscible in water. Such water-miscible, topical, cosmetically acceptable carrier compositions can include those made with one or more appropriate ingredients set forth above but can also include sustained or delayed release carrier, including water-containing, water-dispersable or water-soluble compositions, such as liposomes, microsponges, microspheres or microcapsules, aqueous base ointments, water-in-oil or oil-in-water emulsions, gels or the like.

The disclosure provides a composition for cosmetic use for topical application to the skin. Depending on the method of application, this composition can be provided in any pharmaceutical dosage form normally used in the cosmetic and pharmaceutical fields.

For topical application to the skin, including the scalp, the formulations can have the form of an aqueous, alcoholic or aqueous/alcoholic solution or suspension, of an oily suspension or solution, of an emulsion or dispersion with a liquid or semi-liquid consistency obtained by dispersion of a fatty phase in an aqueous phase (0/W) or vice versa (W/O), of a dispersion or emulsion with a soft consistency, of an aqueous or aqueous/alcoholic or oily (anhydrous) gel, of a free or compact powder to be used as is or to be incorporated in a physiologically acceptable medium (excipient), or also of microcapsules or microparticles, or of vesicular dispersions of ionic and/or nonionic type.

Formulations according to the disclosure can also comprise adjuvants conventional in the cosmetic or pharmaceutical field chosen from hydrophilic or lipophilic gelling agents or thickeners, hydrophilic or lipophilic additives, preservatives, antioxidants (carotenoids), fluids, fragrances, fillers, odor absorbers, electrolytes, neutralizing agents, UV blocking agents, such as sunscreens, film-forming polymers, cosmetic and pharmaceutical active principles with a beneficial effect on the skin or keratinous fibers (such as vitamins) and coloring materials, which may or may not be soluble in the medium. The amounts of these various adjuvants are those conventionally used in the cosmetic field, for example from 0.01% to 20% or from 0.1% to 10% of the total weight of the composition. These adjuvants, depending on their nature, can be introduced into the fatty phase, into the aqueous phase and/or into the lipid vesicles and, in particular, liposomes.

The formulations can also comprise mineral oils (e.g., liquid petrolatum, hydrogenated isoparaffin), vegetable oils (e.g., liquid fraction of karite butter, sunflower oil, soybean oil, wheat germ oil), animal oils (e.g., perhydrosqualene), synthetic oils (e.g., purcellin oil, fatty acid esters), silicone oils or waxes (e.g., linear or cyclic polydimethylsiloxanes, cyclomethicone, phenyl trimethicone), fluorinated oils (e.g., perfluoropolyethers), beeswax, candelilla wax, rice wax, carnauba wax, paraffin wax or polyethylene wax. Fatty alcohols and fatty acids (e.g., stearic acid, linoleic acid, linolenic acid) can be added to these oils and waxes.

The formulations can also include emulsifiers, for example, glyceryl stearate or laurate, polyoxyethylenated sorbitol stearate or oleate, or (alkyl)dimethicone copolyols.

The formulations can also include fluids, for example, of lower alcohols, in particular ethanol and isopropanol, propylene glycol or certain light cosmetic oils (African moringa oil, jojoba, white (mineral) oil, emu oil, rosehip seed oil, avocado oil, almond oil, apricot oil and olive oil).

The formulations can also include hydrophilic gelling agents such as carboxy-vinyl polymers (carbomer), acrylic copolymers, such as acrylate/alkyl acrylate copolymers, polyacrylamides, polysaccharides such as hydroxypropylcellulose, clays and natural gums and mention may be made, as lipophilic gelling agents, of modified clays, such as bentones, metal salts of fatty acids, such as aluminum stearates, hydrophobic treated silica, ethylcellulose and their mixtures.

The formulations may additionally contain anti-fungal or anti-bacterial agents. Anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, oxazalidiinones, streptogramins, and fluoroquinolones. Examples of antibiotic agents include, but are not limited to, linezolid (Zyvax), dalfopristine, quinupristine, Penicillin G (CAS Registry No.: 61-33-6); Methicillin (CAS Registry No.: 61-32-5); Nafcillin (CAS Registry No.: 147-52-4); Oxacillin (CAS Registry No.: 66-79-5); Cloxacillin (CAS Registry No.: 61-72-3); Dicloxacillin (CAS Registry No.: 3116-76-5); Ampicillin (CAS Registry No.: 69-53-4); Amoxicillin (CAS Registry No.: 26787-78-0); Ticarcillin (CAS Registry No.: 34787-01-4); Carbenicillin (CAS Registry No.: 4697-36-3); Mezlocillin (CAS Registry No.: 51481-65-3); Azlocillin (CAS Registry No.: 37091-66-0); Piperacillin (CAS Registry No.: 61477-96-1); Imipenem (CAS Registry No.: 74431-23-5); Aztreonam (CAS Registry No.: 78110-38-0); Cephalothin (CAS Registry No.: 153-61-7); Cefazolin (CAS Registry No.: 25953-19-9); Cefaclor (CAS Registry No.: 70356-03-5); Cefamandole formate sodium (CAS Registry No.: 42540-40-9); Cefoxitin (CAS Registry No.: 35607-66-0); Cefuroxime (CAS Registry No.: 55268-75-2); Cefonicid (CAS Registry No.: 61270-58-4); Cefinetazole (CAS Registry No.: 56796-20-4); Cefotetan (CAS Registry No.: 69712-56-7); Cefprozil (CAS Registry No.: 92665-29-7); Loracarbef (CAS Registry No.: 121961-22-6); Cefetamet (CAS Registry No.: 65052-63-3); Cefoperazone (CAS Registry No.: 62893-19-0); Cefotaxime (CAS Registry No.: 63527-52-6); Ceftizoxime (CAS Registry No.: 68401-81-0); Ceftriaxone (CAS Registry No.: 73384-59-5); Ceftazidime (CAS Registry No.: 72558-82-8); Cefepime (CAS Registry No.: 88040-23-7); Cefixime (CAS Registry No.: 79350-37-1); Cefpodoxime (CAS Registry No.: 80210-62-4); Cefsulodin (CAS Registry No.: 62587-73-9); Fleroxacin (CAS Registry No.: 79660-72-3); Nalidixic acid (CAS Registry No.: 389-08-2); Norfloxacin (CAS Registry No.: 70458-96-7); Ciprofloxacin (CAS Registry No.: 85721-33-1); Ofloxacin (CAS Registry No.: 82419-36-1); Enoxacin (CAS Registry No.: 74011-58-8); Lomefloxacin (CAS Registry No.: 98079-51-7); Cinoxacin (CAS Registry No.: 28657-80-9); Doxycycline (CAS Registry No.: 564-25-0); Minocycline (CAS Registry No.: 10118-90-8); Tetracycline (CAS Registry No.: 60-54-8); Amikacin (CAS Registry No.: 37517-28-5); Gentamicin (CAS Registry No.: 1403-66-3); Kanamycin (CAS Registry No.: 8063-07-8); Netilmicin (CAS Registry No.: 56391-56-1); Tobramycin (CAS Registry No.: 32986-56-4); Streptomycin (CAS Registry No.: 57-92-1); Azithromycin (CAS Registry No.: 83905-01-5); Clarithromycin (CAS Registry No.: 81103-11-9); Erythromycin (CAS Registry No.: 114-07-8); Erythromycin estolate (CAS Registry No.: 3521-62-8); Erythromycin ethyl succinate (CAS Registry No.: 41342-53-4); Erythromycin glucoheptonate (CAS Registry No.: 23067-13-2); Erythromycin lactobionate (CAS Registry No.: 3847-29-8); Erythromycin stearate (CAS Registry No.: 643-22-1); Vancomycin (CAS Registry No.: 1404-90-6); Teicoplanin (CAS Registry No.: 61036-64-4); Chloramphenicol (CAS Registry No.: 56-75-7); Clindamycin (CAS Registry No.: 18323-44-9); Trimethoprim (CAS Registry No.: 738-70-5); Sulfamethoxazole (CAS Registry No.: 723-46-6); Nitrofurantoin (CAS Registry No.: 67-20-9); Rifampin (CAS Registry No.: 13292-46-1); Mupirocin (CAS Registry No.: 12650-69-0); Metronidazole (CAS Registry No.: 443-48-1); Cephalexin (CAS Registry No.: 15686-71-2); Roxithromycin (CAS Registry No.: 80214-83-1); Co-amoxiclavuanate; combinations of Piperacillin and Tazobactam; as well as their various salts, acids, bases, and other derivatives.

Anti-fungal agents include, but are not limited to, terbinafine hydrochloride, nystatin, amphotericin B, griseofulvin, ketoconazole, miconazole nitrate, flucytosine, fluconazole, itraconazole, clotrimazole, benzoic acid, salicylic acid, voriconazole, caspofungin, and selenium sulfide.

In general, the composition of the disclosure is intended to be applied topically and directly to the wound or burn. When the wound is deep, or the burn severe, the composition can be in the form of an ointment, salve or cream that is spread directly onto the wound and then covered with a standard sterile dressing pad or other appropriate dressing material. Alternatively, the ointment, cream or salve of a composition according to the disclosure is applied directly onto the dressing pad or other appropriate dressing material. The pad or dressing material is then placed over the wound or burn with the medicine side contacting the wound or burn. This latter approach works better when applying dressing to severe burns and shallow wounds. For first-degree burns and slight abrasions, the composition may also or alternatively be applied in aerosolized form.

A transdermal patch may be employed to deliver lucuma nut oil compositions to a subject. The patch will include, but is not limited to, a pad material, adhesive, and therapeutic composition. Useful pad materials provide a suitable substrate for the adhesive and are sufficiently strong to withstand removal from the skin, having been secured to the skin by adhesive. In some embodiments, the pad provides a suitable substrate for the formation of apertures therein.

The pad material is sufficiently flexible to provide some comfort to the subject. The flexibility is achieved by elasticity in any one or all axes of the material. Examples of flexible materials include, but are not limited to, cotton cloth, rayon cloth, tetron cloth, nylon cloth or plastic foam. The pad material is preferably pliable to accommodate skin contours, when applied to areas of skin having alterations in surface angles (for example around the nostril skin area). The pad is preferably non-stretchable, namely non-elastic, in the planar axis of the material.

The pad material is also preferably breathable, thereby allowing air to pass through the patch and contact the skin. In some embodiments, however, the pad may not breathable. The pad material is also preferably not permeable to the agent applied to the patch. However, in some embodiments it is preferable that the pad be permeable to the agent. The pad material may also be of a thickness sufficient to provide strength to the pad, but also of a thinness that will be comfortable to the wearer and pliable to contact all skin surfaces.

An adhesive useful in the compositions and methods according to the disclosure is any substance that holds the patch in contact with the skin.

In some embodiments, the agent can be applied to the patch in discrete locations. The pharmaceutical composition is preferably present in an amount and a concentration such that an effective dose of the agent will be applied to the skin over the designated time that the patch remains in contact (e.g., adhered) to the skin. The dosage of agent available to the skin may be altered by altering the density of the discrete applications of the primary agent to the defined surface area of the patch, the cross-sectional area of each application for a defined surface area of the patch, the cavity volume (as measured by the depth and cross-sectional area) of the aperture containing the agent in a defined surface area of the patch, or any combination of these parameters. Thus, where a liner is used as a mask in adding agent to the patch, the greater the depth of the apertures in the liner, the greater the amount of agent available for delivery to the skin. Similarly, the greater the density of apertures, or the cross sectional area of the apertures, the greater the amount of agent available for delivery to the skin.

Delivery of the therapeutic composition to the skin may proceed by a process including, but not limited to, liquefaction upon moisturization of the composition, diffusion of the agent away from the patch or capillary action of the composition from the patch to the skin.

Thus, a pharmaceutical composition of the disclosure is applied to a wound or burn to cover the injured surface completely with, e.g., a one-quarter-inch thickness of the pharmaceutical composition. Preferably, the pharmaceutical composition should be applied within the first twenty minutes following the burn or injury, but the composition may also be applied as soon as possible, and preferably before 12 hours post-injury or post-burn. Dressing-change schedules are dependent on the condition of the wound and those of skill in the art can and will set schedules on a case-by-case basis. For highly contaminated wounds (exhibiting significant amounts of pus), weeping wounds or severe burns, dressing changes may be performed every four to six hours; for other wounds or burns, changes are performed less frequently, sometimes only one or two times per day.

Dressings or patches are advantageously changed three to four times a day. Repeated daily dressing or patch changes are continued until the wound or burn is healed. Healing time varies, depending upon the type and depth of the wound or the severity of the burn.

Pharmaceutical compositions according to the disclosure are effective in the treatment of a large variety of wounds and burns to a mammal, subject or patient in need thereof where bacterial and fungal contamination would ordinarily occur in the absence of treatment. The present medicinal composition can of course also be used to treat burns and wounds in other mammals, such as veterinary animals, including dogs, cats, other household pets, horses, farm animals, and the like.

The data disclosed herein establish another aspect of the disclosure which is the use of LNO as an agent to counter skin aging or to treat skin burns. Accordingly, disclosed herein is a method of promoting skin healing and/or skin regeneration by topical application to the skin of a formulation containing the nut oil extract. The formulation can additionally contain skin health-promoting agents including, for example, antioxidants, retinoids, moisturizers, collagen-promoting agents, sun screens and the like. The formulation can also support the health or repair of the scalp.

The following examples illustrate embodiments of the disclosure. Example 1 describes the preparation of an extract of Pouteria lucuma nut. To the extent that specific materials are mentioned, it is merely for purposes of illustration and is not intended to limit the claims. Example 2 provides experimental results showing that Lucuma nut oil (LNO) induces fibroblast cell migration in an in vitro assay. Example 3 discloses the effect of LNO on in vivo wound healing and regeneration in a mouse skin wound model. Example 4 describes the effect of lucuma nut oil (LNO) on tissue regeneration in a zebrafish tailfin regeneration assay. Example 5 provides a fatty acid methyl ester (FAME) analysis of the composition of LNO. Example 6 discloses Griess assays for NO production in LPS-induced macrophages as a measure of the influence of LNO on inflammatory processes. Example 7 provides an analysis of cytoskeleton remodeling by immunofluorescence staining. Example 8 describes experiments revealing the effect of LNO on bacteria and fungi. Example 9 discloses the effect of LNO on premature cellular senescence.

### Example 1

### Lucuma Nut Oil (LNO) Extraction

In the experiments described in this Example and the following Examples 2-9, media used were LB agar and LB broth, (Fisher Scientific), Potato Dextrose Agar (PDA), and Potato Dextrose Broth (PDB) (Difco). Antibiotics and antifungals used in the experiments included penicillin-streptomycin (Cellgo) and econazole nitrate salt (Sigma # E4632); 96-well plates were from Costar. All other chemical reagents were purchased from Sigma-Aldrich Co.

Wood husks were removed from Pouteria lucuma nuts, i.e., Lucuma nuts, and the resulting nut meat was lyophilized. Lyophilized nuts were finely ground using a coffee grinder. The powder was defatted at a ratio of 1 g nut powder to 100 mL heptane and filtered with a conical paper filter (Whatman). This process was repeated two times. Heptane was removed from the resulting extract using a rotary evaporator (Buchi) followed by a Savant speed vac yielding a yellow oil.

Alternatively, Pouteria lucuma O. Ktze. (syn. Lucuma obvota HBK) nut oil was obtained by extracting 150 g of ground lucuma nut with 2.25 L of heptane for 12 hours. The extraction was done under gentle agitation, protected from light and at room temperature. After extraction, heptane was removed from the extract under vacuum, yielding yellow oil that was stored at −20° C. protected from light.

### Example 2

### Lucuma Nut Oil Induces Fibroblast Migration In Vitro

The capacity of lucuma nut oil extract to influence or affect eukaryotic cell behavior in the form of fibroblast cell migration was assessed using an in vitro cell migration assay standard in the art. Various extracts from the lucuma fruit and nut (seed inside of a fleshy fruit) were examined in cell migration assays in vitro, as cell migration is known to be a feature of wound healing.

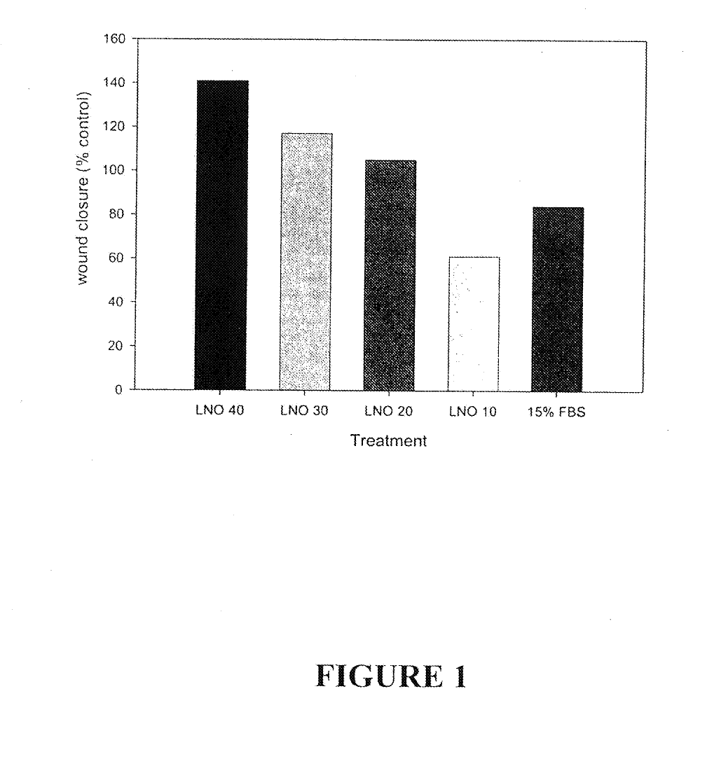

An in vitro fibroblast growth assay showed that lucuma nut extracts increased cell migration. Fibroblasts (i.e., 3T3 fibroblasts) were initially grown to confluence on a glass surface and a scratch was then made in the center of the plate. Plates were treated with either fetal bovine serum (FBS) as a positive control or various concentrations (10-40 μg/ml) of lucuma nut oil (LNO) extracts prepared according to Example 1. Plates were monitored for cell migration into the wound area (% wound closure). LNO increased migration of fibroblast cells in a dose-dependent manner. No activity was found in the fruit or peel extract. The nut meat extracts (referred to as lucuma nut oil or LNO extracts), in contrast, increased cell migration into the wound model in a dose-dependent manner (FIG. 1). Accordingly, LNO promotes cell migration, a feature of the wound healing process.

Human neonatal fibroblasts were maintained in Dulbecco's Modified Eagle Medium (D-MEM)+10% fetal bovine serum (FBS) and were kept in a humidified 37° C. incubator with 5% CO₂. Cells were plated at a density of 1×10⁴ cells per well in a 24-well plate and incubated for 24 hours, or until 100% confluency was reached. Each monolayer was wounded by dragging a pipette tip across the bottom of the well, using a straight edge as a guide (approximately 1.3 mm in width). The media was removed and cells were washed with 500 μL PBS twice to remove detached cells or cell debris. The cells were treated as follows: DMEM+10% FBS+vehicle (0.06% glycerol, positive control), DMEM+vehicle (0.06% glycerol, negative control), DMEM+vehicle (0.06% glycerol)+LNO (60 μg/mL), DMEM+vehicle (0.06% glycerol)+LA (24 μg/mL) and DMEM+vehicle (0.06% glycerol)+OA (18 μg/mL). Photos were taken of each well to measure the initial area of the wound. After 24 hours incubation, wells were photographed again. The scratched area was measured using Acrobat Photoshop CS software, and percentage of wound closure at each time point was derived by the following formula: (1−[current wound size/initial wound size])×100.

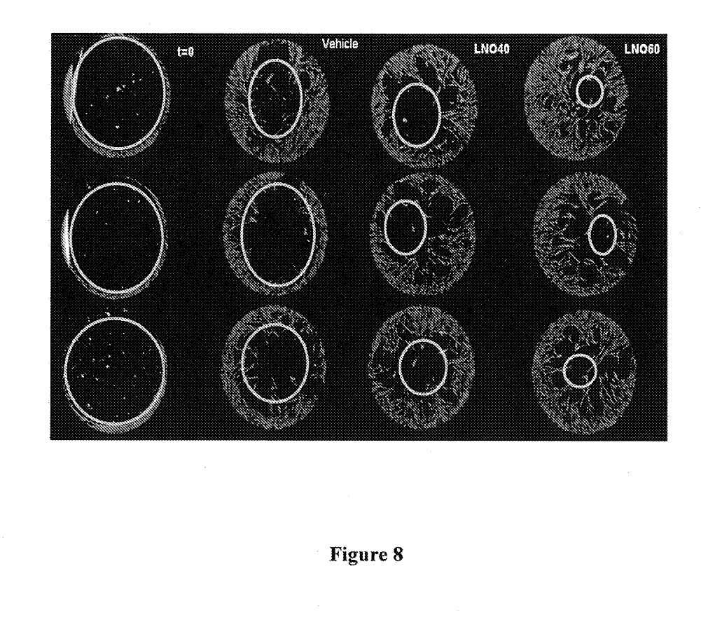

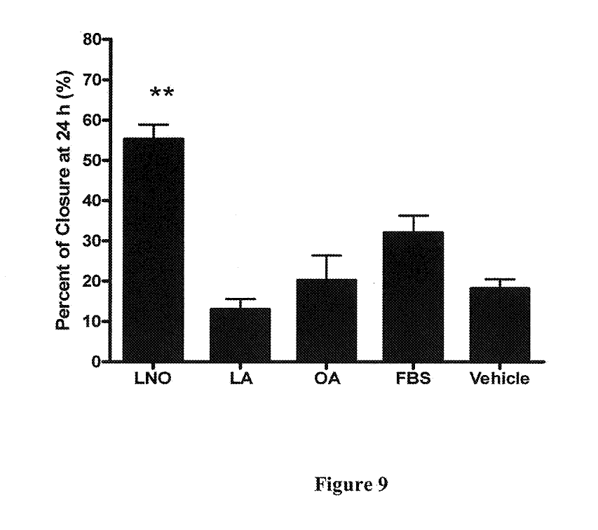

The effect of LNO on fibroblast migration was also assessed by the Oris™ Cell Migration Assay from Platypus Technologies according to the manufacturer's instructions. Briefly, human neonatal fibroblasts were maintained in Dulbecco's Modified Eagle Medium (D-MEM)+10% fetal bovine serum (FBS) and kept in a humidified 37° C. incubator with 5% CO₂. Then 25,000 cells/well were seeded into a 96-well plate and allowed to adhere to the collagen I-coated plate for 24 hours, serum-starved for 24 hours, and treated with LNO or vehicle for 72 hours. The stoppers were then removed from the 96-well plate to create a 2.0 mm detection zone, cells were labeled with Calcein AM 4.0 μM and migration into the exclusion zone was measured by fluorescent microscopy. The results demonstrated that LNO promoted migration of human fibroblast into the exclusion zone (FIG. 8). The effects of LNO versus pure oleic (OA) and linoleic (LA) acids were compared on scratched fibroblasts. LA and OA are the major FAs of LNO. These results demonstrated that the wound-healing effect of LNO cannot be explained by the sole presence of LA or OA, as equivalent concentrations of these pure FAs were not able to reproduce the effect of LNO in cell migration (FIG. 9).

### Example 3

### In Vivo Wound Healing and Regeneration-Skin Wound

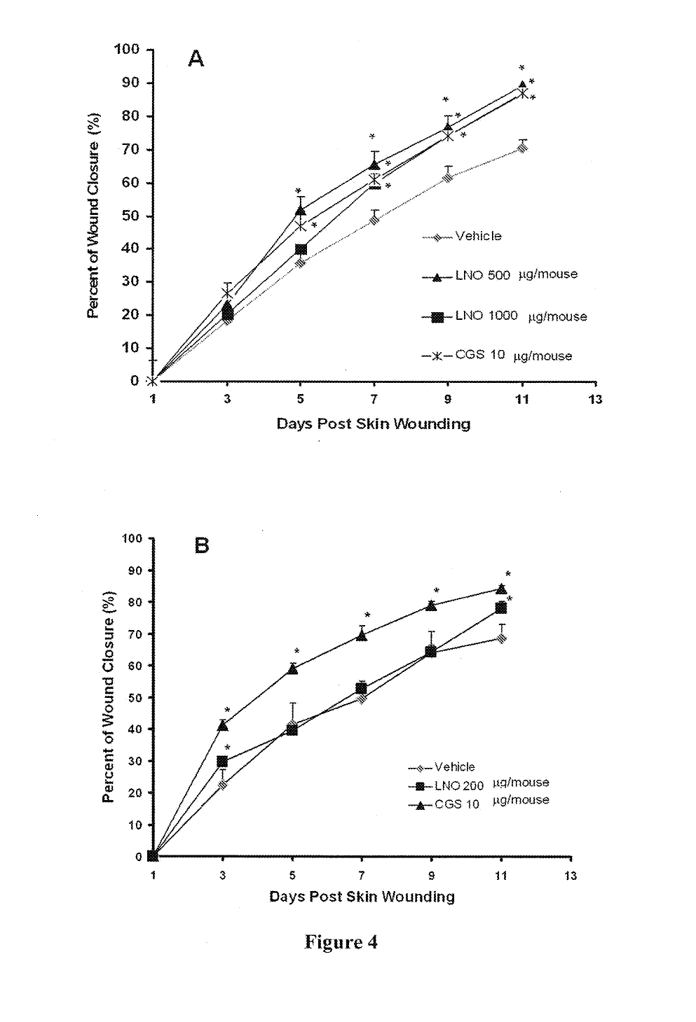

The LNO extract of Example 1 was further used in two in vivo models to assess wound healing and regeneration. In the CD-1 mouse excision wound healing assay, LNO decreased the time required for wound closure (FIG. 4A). In this experiment, a 12 mm skin punch was used to create a wound in mice and the mice were then divided into three groups, i.e., a negative control group, an experimental group and a positive control group. Immediately following wounding, the wounds were treated for ten days with either vehicle (0.5% carboxymethylcellulose in PBS), lucuma nut oil (LNO) (200 μg/mouse) in vehicle, or the positive control CGS-21680 (CAS120225-54-9, a specific adenosine A_{2A} receptor agonist offered commercially by Tocris; see U.S. Pat. No. 4,968,697), also in vehicle. The median time required for wound closure (CT₅₀) is shown in FIG. 4A. The data establish that LNO decreased the time required for wound closure (CT) versus vehicle control.

Wound healing assays were performed by MDS Pharma Services, Taiwan, MDSPS Testing Code 1115924, according to the protocol describe elsewhere (Kapoor, et al., J. Clin. Invest., 118:3279-3290 (2008); Montesinos, et al., J. Exp. Med., 186:1615-1620 (1997), each description incorporated herein by reference). Briefly, groups of 5 male CD-1 mice weighing 22±2 g were used for this assay. Under ether anesthesia, the shoulder and back region of each animal was shaved and a sharp punch (internal diameter 12 mm) was used to remove the skin, including the panniculus carnosus and adherent tissues. The wounded area was traced onto clear plastic sheets on days 3, 5, 7, 9 and 11 and quantitated by using an Image Analyzer (Life Science Resources VISTA, Version 30). Adenosine agonist CGS, LNO or vehicle were topically applied immediately after injury and once daily thereafter for a total of 10 consecutive days.

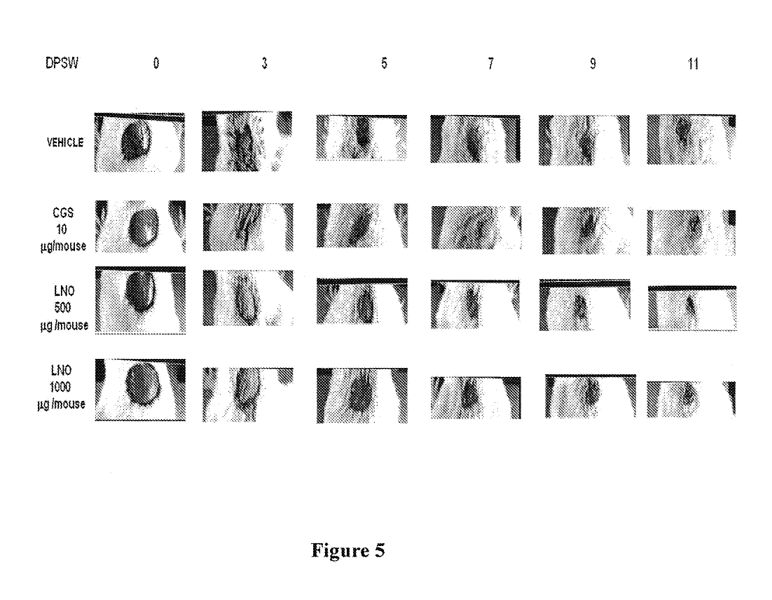

Topical application of LNO onto the dermal wounds of DC-1 mice showed the significant wound-healing properties of LNO. Treated animals showed almost 100% wound closure after 10 days of topical administration of LNO (FIGS. 4 and 5), which was significantly higher than that of the vehicle and comparable to the positive control. The pronounced in vivo wound-healing effect of LNO (Table 1) is explained by its unique ratio of FAs and by the potentiating effects of several anti-inflammatory FAs contained in the LNO. Wound healing or wound closure data obtained in these experiments are presented in Table 1.

TABLE 1
Dermal wound closure expressed as half-closure time (CT₅₀)

| Treatment | CT₅₀^{a} ± SE^{b} (days) |
|---|---|
| Vehicle | 7.58 ± 0.4 |
| CGS 10 μg/mouse | 6.04 ± 0.2* |
| LNO 200 μg/mouse | 6.84 ± 0.2 |
| LNO 500 μg/mouse | 5.86 ± 0.3* |
| LNO 1000 μg/mouse | 6.34 ± 0.1* |

^{a}Time at which 50% of the skin wound is closed.
^{b}Standard error
*P < 0.05, vs. vehicle (One-way ANOVA followed by Dunnett's test)

### Example 4

### In Vivo Wound Healing and Regeneration-Tailfin Primordial Amputation

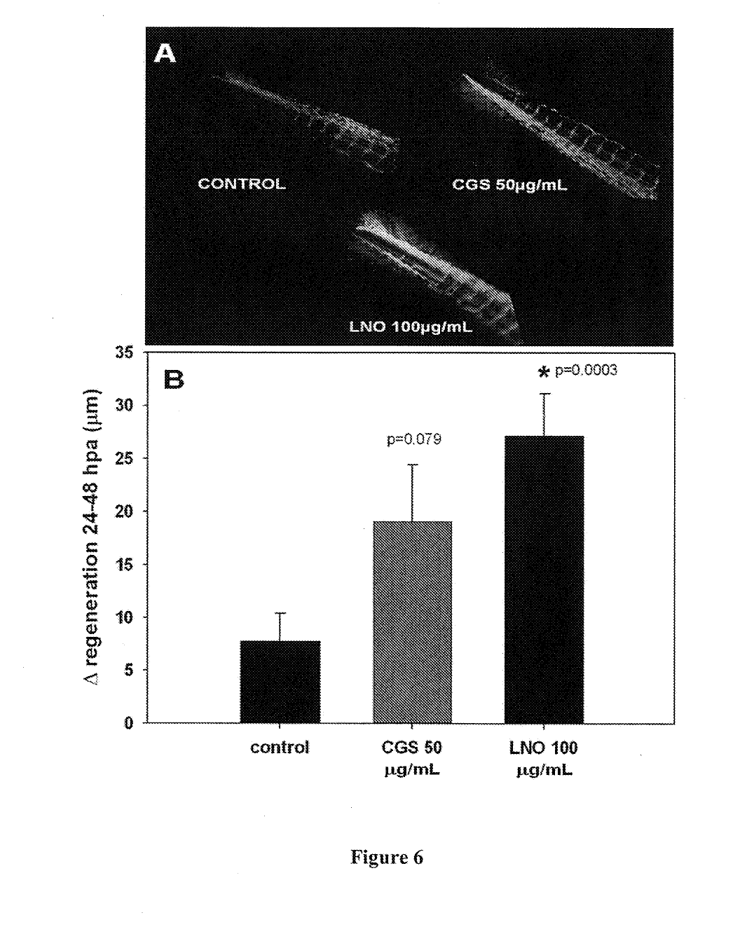

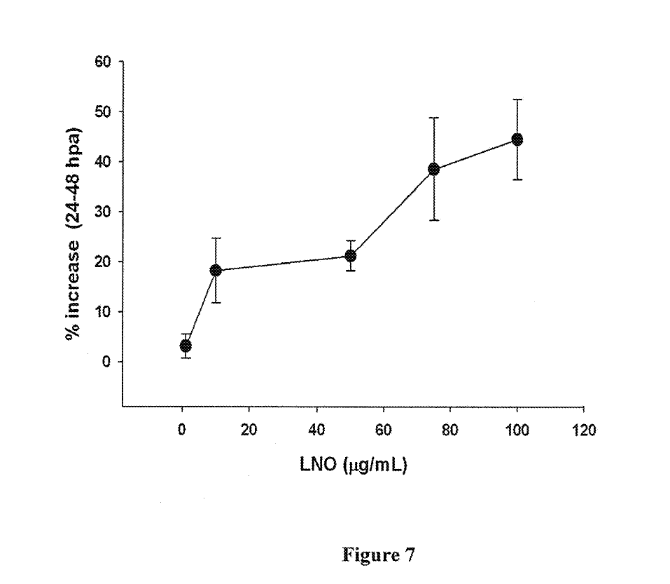

A tailfin model of in vivo wound healing and regeneration was also used to assess the ability of a lucuma nut oil extract to promote appropriate wound healing, and the results of these assays also provided information on the angiogenic properties of LNO. In this transgenic model, the fli1 promoter drives expression of green fluorescent protein (GFP) in all the vascular endothelial cells, which allows tracking of wound healing in real time by direct observation of angiogenesis. Forty-eight-hour-old or five-day-old zebrafish larvae (AB strain) were anesthetized in 0.04% 3-amino benzoic acid ethylester (tricaine; MS-222) and placed in 2% agar Petri dishes. Surgical blades were used to amputate tailfin primordia just posterior to the notochord. Larvae were placed in 24-well plates containing either water (control), CGS-21680 (50 μg/mL; CAS120225-54-9) or LNO (10-100 μg/mL in water) and incubated at 28° C. Primordia re-growth was measured using fluorescence microscopy and image analysis of fluorescent endothelial cells. For this assay, the fin fluorescent area was normalized against the total fin area. Photographs of regenerating tailfins were taken at 24 and 48 hours post-amputation using a Scion CFW-1310C color digital camera with an Olympus SZ4060 zoom stereo microscope. The effects of LNO were analyzed and compared to the positive control CGS-21680 (2-p-(2-Carboxyethyl)phenethylamino-5′-N-ethylcarboxamidoadenosine). The results, shown in FIG. 6B, establish that LNO increased the amount of regenerative tissue versus both non-treated and positive control larvae. These effects were dose-dependent (FIG. 7). These results are in agreement with the data from CD-1 mice and indicate that LNO is not only a wound healing agent, it is also a promoter of the formation of new blood vessels in wounded areas.

Taken together, the experimental results presented in Examples 3 and 4 indicate that the nut oil extracted from lucuma has significant activity in enhancing wound healing processes.

### Example 5

### GC-MS Analysis of LNO

For fatty acid methyl ester (FAME) analysis, a 100 μL aliquot of LNO was added to 1 mL methanolic HCl and heated for 1 hour at 100° C., gently shaken every 15 minutes for 1 minute. Vials were cooled in an ice bath and 2 mL hexane added. Samples were gently stirred in a tabletop incubator for 5 minutes at 120 rpm and then centrifuged for 3 minutes at 2100×g. The organic phase was transferred to a new vial, dried under a stream of nitrogen and reconstituted in 100 μL of methylene chloride. This extract was diluted 1:10 twice for further analysis. A 1 μL aliquot of the diluted sample was injected into a heated (250° C.) split (50:1) injector and separated on an SGE BPX70 (highly polar cyanopropyl polysilphenylsiloxane) capillary column (60 m×0.25 mm ID×0.25 μm film). Oven conditions were: 100° C. hold 2 minutes, ramp 5° C./minute to 150° C. hold 10 minutes; ramp 2° C./minute to 200° C. hold 8 minutes, ramp 5° C./minute to 240° C. hold 1 minute. The carrier gas was helium flowing at 1 mL/minute. Mass spectra from 40 to 550 m/z were acquired using a dual-stage quadrupole with source at 250° C. and +EI ionization of 70 eV. A 37-component food industry fatty acid mixture at 100 μg/mL each was injected for verification. Results are presented in Table 2.

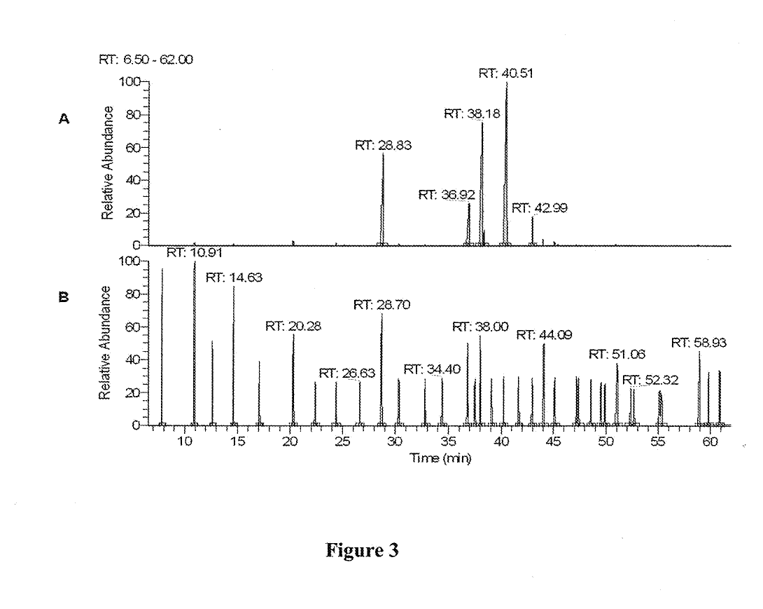

The heptane extract from lucuma nut yielded 2.6% LNO from ground lucuma nut powder. GC/MS analyses of fatty acid methyl esters (FAMEs) indicated that the most abundant FAs in LNO were linoleic (C18:2), oleic (C18:1), palmitic (C16:0), stearic (C18:0), and γ-linolenic acid (C18:3) (Table 2, FIG. 3). In addition, there were five as yet unidentified FAs. None of the unknowns contributed significantly to fatty acid composition of the LNO. Several of the FAs from LNO have been reported as potentially relevant in maintaining healthy skin and in improving lipid metabolism in humans. Linoleic and oleic acids have been reported as wound healing agents potentially acting as modulators of skin inflammation. Another bioactive molecule detected in LNO was γ-linolenic acid (GLA). Both GLA and its metabolites can prevent uncontrolled inflammation damage to healthy cells that severely impair the wound healing process.

TABLE 2
Chemical composition of lucuma nut oil

| % | | Fatty Acid Methyl | | |
|---|---|---|---|---|
| Area¹ | Fatty Acid | Ester (FAME) | Carbon² | RT³ |
| 38.85 | linoleic acid | methyl linoleate | 18:2 | 40.51 |
| | | (cis-9, 12) | | |
| 27.85 | oleic acid | methyl oleate (cis-9) | 18:1 | 38.18 |
| 18.63 | palmitic acid | methyl palmitate | 16:0 | 28.83 |
| 8.94 | stearic acid | methyl stearate | 18:0 | 36.92 |
| 2.93 | γ linolenic acid | methyl γ-linolenate | 18:3 | 42.99 |
| | | (cis-6, 9, 12) | | |
| 0.61 | arachidic acid | methyl arachidate | 20:0 | 44.01 |
| 0.42 | myristic acid | methyl myristate | 14:0 | 20.24 |
| 0.34 | eicosenoic acid | methyl eicosenoate | 20:1 | 45.09 |
| | | (cis-11) | | |
| 0.24 | pentadecanoic acid | methyl pentadecanoate | 15:0 | 24.34 |
| 0.17 | palmitoleic acid | methyl palmitoleate | 16:1 | 30.29 |
| | | (cis-9) | | |
| 0.14 | decanoic acid | methyl decanoate | 10:0 | 10.90 |
| 0.11 | heptadecanoic acid | methyl heptadecanoate | 17:0 | 32.76 |
| 0.1 | lauric acid | methyl laurate | 12:0 | 14.60 |
| 0.1 | behenic acid | methyl behenate | 22:0 | 50.90 |
| 0.08 | ? | ? | ? | 45.42 |
| 0.07 | ? | ? | ? | 25.16 |
| 0.07 | eicosadienoic acid | methyl eicosadienoate | 20:2 | 47.17 |
| | | (cis-11, 14) | | |
| 0.05 | erucic acid | methyl erucate (cis-13) | 22:1 | 52.21 |
| 0.04 | ? | ? | ? | 46.75 |
| 0.04 | heneicosanoic acid | methyl heneicosanoate | 21:0 | 47.33 |
| 0.04 | docosadienoic acid | methyl docosadienoate | 22:2 | 52.65 |
| | | (cis-13, 16) | | |
| 0.04 | lignoceric acid | methyl lignocerate | 24:0 | 58.77 |
| 0.03 | ? | ? | ? | 55.58 |
| 0.02 | octanoic acid | methyl octanoate | 8:0 | 7.85 |
| 0.02 | ? | ? | ? | 60.02 |
| 0.01 | ? | ? | ? | 47.50 |
| 0.01 | ? | ? | ? | 47.88 |
| 0.01 | eicosapentoic acid | methyl eicosapentanoate | 20:5 | 55.17 |
| | | (cis, 5, 8, 11, 14, 17) | | |
| 0.01 | ? | ? | ? | 63.43 |

¹All peak areas integrated and summed. Area of each FAME was divided by summed peak area of all FAMEs to calculate % area.
²Total carbon number and unsaturation (if any). e.g. 16:0 represents the completely saturated palmitic acid.
³RT = retention time in minutes (see FIG. 3).

### Example 6

### LNO Decreases NO Production in Macrophages

Murine RAW264.7 macrophages were obtained from the American Type Culture Collection (ATCC). RAW264.7 cells were cultured in DMEM, supplemented with 10% heat-inactivated fetal bovine serum, and maintained in a 37° C. humidified incubator containing 5% CO₂. The RAW264.7 cells were plated at a density of 0.4×10⁵ cells/well, in 24-well plates. Cells were pre-treated for 2 hours with LNO prior to incubation with LPS at 1 μg/mL for 8 hours. Cells were subcultured by scraping plates when cells reached 90% confluency.

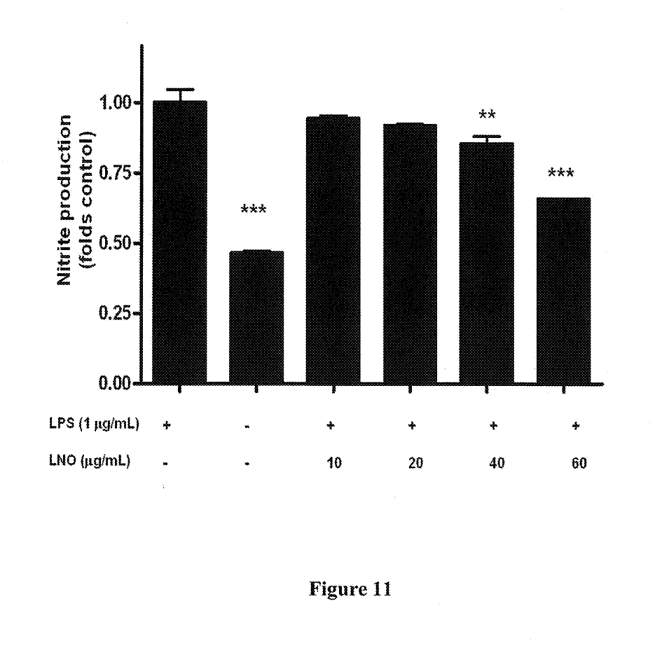

The Griess assay was performed to indirectly measure nitric oxide production by LPS-induced macrophages. The Griess assay, which provides a measure of nitrite in the culture medium, was performed according to the protocol described elsewhere (Park, et al., J. Leukoc. Biol., 54:119-124 (1993)), with minor modifications. Briefly, RAW macrophages were plated at a minimum density of 0.4×10⁵ cells/well in a 24-well plate and grown for 24 hours. Stock solutions of LNO and triptolide (positive control) were added to the medium 2 hours before LPS stimulation. Cells were stimulated with 1 μg/mL LPS (Sigma-Aldrich, Inc., St. Louis, Mo.). After 8 hours, conditioned media (50 μL) was removed and immediately mixed with 50 μL of Griess reagent (10% sulfanilamide, 1% naphthaleneethylenediamine dihydrochloride in 5% H₃PO₄). After incubation for 15 minutes at room temperature in darkness, samples were read at 540 nm using a BIOTEK micro-plate spectrophotometer. LNO did not show any cytotoxic effect at the concentrations used in our studies as evaluated by the MTT viability assay. LNO produced a moderate but significant decrease in the concentration of nitric oxide (FIG. 11). The decrease in nitric oxide production after LNO application was dose dependent. Taken together, the results from the in vivo mice wounding assay, the transgenic zebrafish larvae tailfin regeneration assay, and the LPS-stimulated macrophage behavior, establish that LNO modulates the inflammatory process in a manner that accelerates the wound healing process.

### Example 7

### Immunofluorescence Staining

Cells were seeded into 24-well plates containing glass coverslips, serum-starved for 24 hours, and treated with LNO 60 μg/mL or vehicle for 24 hours. For staining of stress fibers and vinculin, cells were fixed with ice cold 4% paraformaldehyde in PBS for 15 minutes and permeabilized with ice cold 0.5% (vol/vol) Triton X-100 in PBS for 5 minutes at room temperature. Cells were washed with PBS and then permeabilized for 2-5 minutes. Cells were washed with PBS and then blocked with 3% (wt/vol) BSA in PBS for 1 hour. Vinculin mAb FITC-Conjugate (Sigma, Cat F7053) 1/200 or Rhodamine Phalloidin 140 nM (Cytoskeleton Inc), diluted in 3% BSA (wt/vol) in PBS was applied, and the cells were incubated overnight at 4° C. Cells were washed again for at least 20 minutes with PBS and then mounted in 80% glycerol PBS.

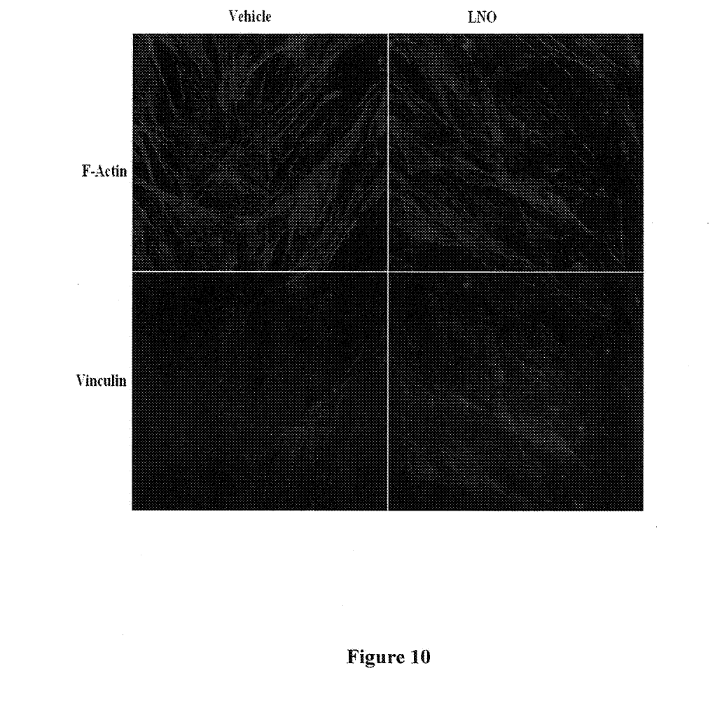

The results showed that LNO at 60 μg/mL induced a mild decrease in stress fiber formation and increased the expression of vinculin (FIG. 10). These results are consistent with the finding that LNO increased cell migration by increasing vinculin expression and mildly decreasing stress fiber formation in fibroblasts, thus favoring cell motility.

### Example 8

### LNO Effects on Bacteria and Fungi

The bacteria and fungi studied were Escherichia coli DH5α (ATCC # 47093), Staphylococcus aureus (ATCC # 12600), Enterobacter aerogenes (ATCC # 13048), Mycobacterium rhodococcus DK17, and Saccharomyces cerevisiae (ATCC # 201459). The medium for the growth of all bacteria was LB agar at 37° C. The medium for the growth of S. cerevisiae was Potato Dextrose Agar (PDA). E. coli and S. aureus were grown on LB agar plates at 37° C. E. aerogenes and M. rhodococcus DK17 were grown on LB agar plates at 30° C. S. cerevisiae was grown on PDA plates at 30° C. For inhibition experiments, LB broth was used for all bacteria and Potato Dextrose Broth (PDB) was used for testing S. cerevisiae.

The minimal inhibitory concentration (MIC) values were determined by broth dilution assay, using a method modified from Jones et al., Susceptibility tests: microdilution and macrodilution broth procedures, in Manual of Clinical Microbiology, 4th ed. American Society for Microbiology, A. B. E. H. Lennette, W. J. Hausler, Jr., and H. J. Shadomy, Editor. 1985, American Society for Microbiology, Washington, D.C. and C L S I Ferraro, Methods for dilution antimicrobial susceptibility tests for bacteria that grow aerobically. Vol. 17. 1997: Natl. Comm. Clin. Lab. Stds, Wayne. The descriptions are incorporated herein by reference The above-identified microorganisms were grown for 24 hours in broth cultures. The MIC was defined as the lower concentration of the compound to inhibit growth of microorganisms compared to the growth of the untreated control. Media inoculated with the preferred microorganism (5×10⁵ cells/mL) was added to each well of a 96-well plate containing 10-1000 μg/mL of LNO. The plate was then incubated at 30° C. or 37° C. (depending on the microorganism) for 24 hours. The inhibitory effect of LNO on the growth of the tested organism was evaluated using a Multi-Detection Microplate Reader (Synergy HT, Biotek) at 600 nm (absorbance).

All bacteria and fungi were tested in triplicate over the range of 10 μg/mL to 1000 μg/mL LNO concentrations. No significant inhibitory effect was observed up to the concentration of 1000 μg/ml of LNO.

### Example 9

### LNO Effects on Premature Cellular Senescence

During cellular senescence, cells display several changes in their phenotype. Among other characteristics, alterations in nuclear morphology are characteristic of senescent cells. Globular, folded, fragmentized or oversized nuclei are frequently found in senescent cells. These anomalous morphological features are hallmarks of the aging process in senescent cells. In the experiments described in this Example, the effect of LNO on cellular senescence was assessed by studying the changes in nuclear morphology of H₂O₂-induced senescent fibroblasts.

Human neonatal fibroblasts (Cascade Biologics Catalog Number: C-004-5C) were maintained in Dulbecco's Modified Eagle Medium (DMEM)+10% fetal bovine serum (FBS) and were kept in a humidified 37° C. incubator with 5% CO₂ For induction of premature senescence, exponentially growing fibroblasts were inoculated at a cell density of 5×10⁵ in 50 mm dishes. Three days after seeding, the cells were treated with the culture medium containing H₂O₂ (100 μM), H₂O₂ (100 μM)+LNO 60 μg/mL, or just culture medium for 1 hour. After the treatments, fibroblasts were trypsinized, passaged normally, and incubated in fresh complete medium for another 7 days. Cells were then fixed in ice-cold methanol for 5 minutes and stained with Acridine orange. Fluorescent microphotographs of cell nuclei were taken with an Olympus 1×51 microscope coupled to a CWF 1310C camera (Scion Corporation).

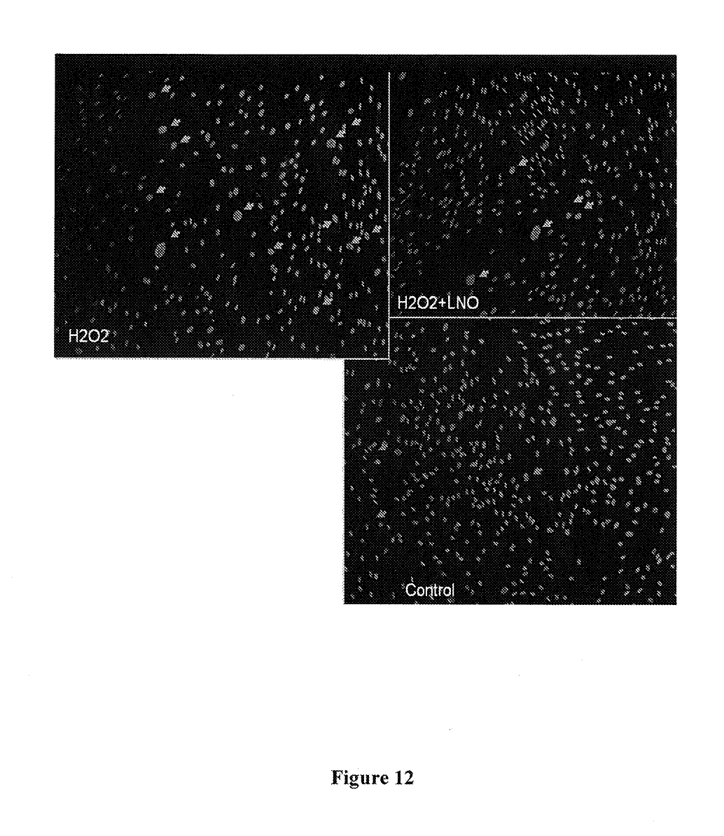

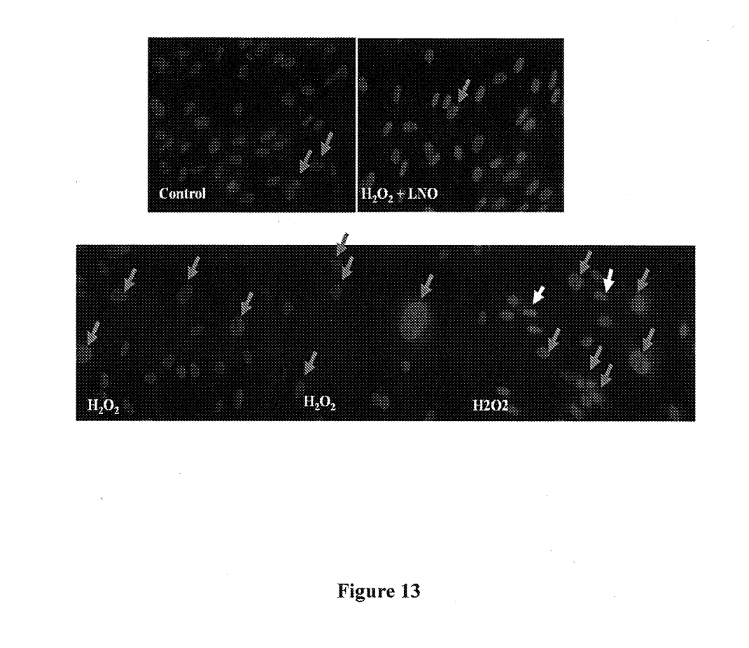

By fluorescence microscopy, non-senescent cells (control) showed increased cell density, classical elongated nuclear shape and homogeneous nuclear size. Conversely, cells treated with H₂O₂ displayed lower cell density and abnormal nuclear morphology, such as oversized and folded nuclei, which are characteristic features of the senescent phenotype (FIGS. 12 and 13). Cells treated with H₂O₂+LNO showed fewer cells with senescent nuclear abnormalities compared with H₂O₂-treated cells (FIGS. 12 and 13, orange arrows). Thus, the results of these experiments show that LNO prevents dermal fibroblasts from entering into premature senescence.

LNO is disclosed herein as a new source of compounds, including natural fatty acids (FAs) consisting mainly of linoleic acid, oleic acid, palmitic acid, stearic acid and γ-linolenic acid. LNO displayed a pronounced wound-healing effect, both in mice and transgenic zebrafish larvae. Fatty acids (FAs), particularly polyunsaturated fatty acids (PUFAs), play an important role in inflammatory processes, as they are metabolic precursors of several key mediators of inflammation, such as eicosanoids, lipoxins and resolvins.

Wound closure is a highly coordinated sequence of molecular events involving inflammation, fibroblast migration, re-epithelialization, extracellular matrix (ECM) remodeling bacterial and fungal growth, and angiogenesis. Several cytokines and nitric oxide are important mediators of this process. Angiogenesis is known to be important in the formation of granulation tissues and supply of oxygen to wounded tissues. Another important aspect in the treatments of cutaneous wounds is the possibility of bacterial and fungi infections, especially in immunocompromised individuals. Infected skin wounds are frequently associated with delayed wound closure, prolonged inflammation and severe scar formation. Colonized or infected wounds tend to develop an abnormally prolonged inflammatory state which generates large amounts of nitric oxide. Disclosed herein are the results of experiments establishing that LNO, a composition comprising a mixture of fatty acids obtained as an extract from the nut of a South American fruit, promotes wound healing. The effect of LNO on the wound healing process was investigated, including investigations of such relevant events as fibroblasts migration, revascularization, bacterial and fungal growth and nitric oxide production. In a transgenic zebrafish model, LNO not only accelerated wound healing, it also increased new GFP-positive endothelial cells. This finding indicates that FAs in LNO increase vessel sprouting, thus improving perfusion of wounded areas. This effect was not found using pure LA and OA, the major components of LNO.

The data from cell migration experiments led to the conclusion that the in vivo wound healing effect of LNO is related to the increase in the migration capacity of fibroblasts. As focal adhesions are important for fibroblast migration and adhesion, changes in the expression of focal adhesion proteins in fibroblasts treated with LNO were expected. In fact, it has been disclosed that fibroblasts from wounded areas increase expression of vinculin and the increased expression is correlated with a higher migratory capacity (van Beurden, et al., Wound Repair Regen., 14:66-71 (2006); van Beurden, Eur. J. Oral Sci., 113:153-158 (2005)). During cell migration, vinculin and stress fibers play a key role in the integrin-mediated interaction of fibroblasts with the ECM. Integrins are key players in fibroblast interaction with ECM and with other cells. During the wound healing process, integrins nucleate three distinct matrix adhesions in fibroblastic cells, i.e., focal complexes, focal adhesions and fibrillar adhesions. Focal complexes contain paxillin, vinculin, α-actinin, talin, focal adhesion kinase (FAK) and tyrosine-phosphorylated proteins. Fibrillar adhesions are located centrally in migrating cells and contain fibronectin and tensin. β1 integrins can translocate from focal complexes to focal adhesions and ultimately fibrillar adhesions, indicating that a precursor-product relationship between the three types of matrix adhesion is necessary for optimum cell migration. A mild disassembly of stress fibers and increased expression of vinculin in focal adhesion has been shown to be important for cell migration. The data indicate that the wound healing effect of LNO observed in mice and transgenic zebrafish larvae is produced by increasing fibroblasts migration, promoting angiogenesis and modulating nitric oxide production. Further, LNO has been shown herein to antagonize cellular senescence. Therefore, LNO contains a unique combination of natural, pharmacologically active compounds, e.g., FAs, making the total extract more effective than, e.g., the individual FAs it contains.

Numerous modifications and variations of the disclosure are possible in view of the above teachings and are within the scope of the claims. The above-described embodiments are not intended to limit the claims in any way. The entire disclosure of all publications cited herein are hereby incorporated by reference.

What is claimed is:

1. A method of preparing an extract of Pouteria lucuma nut comprising:
(a) grinding a Pouteria lucuma nut to a powder;
(b) extracting nut oils from the powder with fluid; and
(c) removing the fluid to obtain an oil.

2. The method of claim 1, further comprising preparing a pharmaceutical formulation comprising the nut oil.

3. The method of claim 1, further comprising preparing a cosmetic formulation comprising the nut oil.

4. The method of claim 1, wherein the fluid is selected from the group consisting of heptane and hexane.

5. The method of claim 4, wherein the fluid is removed by evaporation.

6. The method of claim 4, wherein the ratio of nut powder to fluid is between 1 g:10 mL and 1 g:100 mL, weight to volume.

7. An extract of Pouteria lucuma nut comprising the nut oil.

8. A cosmetic formulation comprising the extract of claim 7.

9. The formulation of claim 8, further comprising an antibiotic.

10. The formulation of claim 8, further comprising a skin health-promoting agent.

11. The formulation of claim 10, wherein the skin health-promoting agent is selected from the group consisting of Shea butter, an antioxidant, a retinoid, a moisturizer, a collagen-promoting agent, a sun screen, an alpha hydroxy acid, jojoba oil, a silk extract, isopropyl palmitate, methyl paraben and propyl paraben.

12. The formulation of claim 11, wherein the antioxidant is selected from the group consisting of vitamin A, vitamin C and vitamin E.

13. The formulation of claim 8, formulated as a cream, lotion, gel, paste, or spray for application to skin or scalp.

14. A method of wound healing comprising administering an effective dose of a Pouteria lucuma nut extract.

15. A method of inducing cell migration comprising contacting a cell with the extract of claim 7.

16. The method of claim 15, wherein the cell is a fibroblast.

17. A method of enhancing tissue regeneration comprising contacting a tissue capable of regeneration with Pouteria lucuma nut oil, wherein the tissue regenerates at a rate that is greater than is exhibited by the tissue in the absence of the nut oil.

18. A method of promoting skin healing comprising contacting the skin with a pharmaceutical composition comprising the extract of claim 7.

19. A method of reducing skin senescence comprising contacting the skin with a pharmaceutical composition comprising the extract of claim 7.

20. A method of healing a skin burn comprising contacting the skin burn with a pharmaceutical composition comprising the extract of claim 7 in an amount effective to promote skin healing in a patient in need thereof.

21. The method of claim 20, wherein the pharmaceutical composition further comprises an antibiotic.

22. A method of reducing, eliminating or preventing a skin condition selected from the group consisting of acne, skin aging and wrinkled skin comprising contacting skin with a cosmetic formulation comprising the extract of claim 7.

23. The method of claim 22 wherein the skin condition is wrinkled skin.

24. The method of claim 22 wherein the skin condition is skin aging.

25. The method of claim 22 wherein contacting the skin with the extract of claim 7 results in a relative increase in the expression of a protein selected from the group consisting of collagen and elastin in a skin cell.

26. The method of claim 22, wherein the cosmetic formulation further comprises an anti-acne composition.

27. The method of claim 26, wherein the anti-acne composition is selected from the group consisting of salicylic acid, benzoyl peroxide, glycolic acid, sulphur, retinoic acid, peat water, resorcinol, silt, peat, permethin, azelaic acid, clindamycin, adapalene, erythromycin and sodium sulfacetamide.

28. The method of claim 27, wherein the cosmetic formulation further comprises an antibiotic.

29. A transdermal patch comprising a pad material having an upper surface and a lower surface, an adhesive on the lower surface, and a pharmaceutical composition comprising the extract of claim 7 on the lower surface.

30. The patch of claim 29, further comprising an antibiotic on the lower surface.
